(19) United States
(12) Patent Application Publication
(10) Pub. No.: US 2016/0262847 A1
(43) Pub. Date: Sep. 15, 2016

(54) DENTAL OR SURGICAL HANDPIECE

(71) Applicant: SycoTec GmbH & Co. KG, Leutkirch im Allgau (DE)

(72) Inventors: Martin Rickert, Bad Wurzach (DE); Karl Mack, Altusried (DE); Carolin Rickert, Bad Wurzach (DE)

(21) Appl. No.: 15/065,823

(22) Filed: Mar. 9, 2016

(30) Foreign Application Priority Data
Mar. 9, 2015 (DE) 10 2015 103 432.8
Jul. 20, 2015 (DE) 10 2015 111 757.6

(51) Int. Cl. A61B 90/98; A61C 17/02; A61C 1/08; A61C 1/14

(52) U.S. Cl. CPC A61B 90/98; A61C 1/141; A61C 17/0202; A61C 1/088; A61B 2090/306

(57) ABSTRACT

The invention refers to a dental or surgical handpiece comprising a head, which serves to receive a tool, and a connector sleeve, which is intended for coupling the handpiece to a drive motor, wherein the head and the connector sleeve are connected by a connecting piece and the connecting piece accommodates a drivetrain which serves to transmit rotational energy from the connector sleeve into the head for driving the tool, characterized in that the handpiece has a RFID transponder and a plastic casing delimiting the handpiece, and the RFID transponder is protected by the plastic casing.

### FIELD OF THE INVENTION

The invention relates to a dental or surgical handpiece. This has a head which serves to receive a tool. It also has a connector sleeve which is intended for coupling the handpiece to a drive motor. The head and the connector sleeve are connected by a connecting piece. The connecting piece supports a drivetrain which serves to transmit rotational power from the connector sleeve into the head for driving the tool.

### BACKGROUND OF THE INVENTION

Such handpieces are typically used in order to provide tools for various treatments to the human or animal body. Such a tool may, for example, be a drill for teeth or a cutting tool for surgical intervention. Known handpieces are typically made of metal, in particular with a metallic outer skin.

In the medical field there is, in principle, a particularly high demand for hygiene and sterility of instruments. Therefore it is typically necessary for the tools and handpieces which have been used to be cleaned and disinfected or sterilized regularly, for example after every use or at the end of a day. This can take place, for example, in an autoclave.

In the event of an official inspection or also in the event of an infection occurring in the patient it may be necessary to demonstrate the correct cleaning and sterilization of a tool or of a handpiece. In this connection such procedures are regularly documented, which typically takes place manually and necessitates considerable expenditure.

### SUMMARY OF THE INVENTION

It is therefore an object of the invention to provide a handpiece which can be handled more easily, for example, with regard to the documentation.

This is achieved according to the invention by a handpiece according to claim 1. Advantageous embodiments are disclosed, for example, by the subordinate claims.

The invention relates to a dental or surgical handpiece. This has a head which serves to receive a tool. It also has a connector sleeve which is intended for coupling the handpiece to a drive motor. The head and the connector sleeve are connected by a connecting piece. The connecting piece supports a drivetrain which serves to transmit rotational power from the connector sleeve into the head for driving the tool.

According to the invention, it is provided that the handpiece has a RFID transponder and a plastic casing delimiting the handpiece, and the RFID transponder is protected by the plastic casing.

RFID designates a technology for transmitter-receiver systems for automatic and contactless identification and the location of objects with radio waves.

A RFID system consists of a RFID transponder (also referred to as a radio tag) which is located on or in the object and contains an identifying code. The RFID transponder co-operates with a reading device.

The coupling takes place by high-frequency radio waves generated by the reading device. Thus not only is data transferred, but also the transponder is supplied with power. In this case, the data transmission takes place, for example, unidirectionally from the transponder to the reading device (using the transmitted power), or also bidirectionally, where data is also written to the transponder.

An automated recognition of the handpiece is possible by means of the RFID transponder. Thus, for example, in the case of cleaning or sterilizing procedures the respective device, for example an autoclave, can automatically recognize the respective handpiece. For this purpose data, such as, for example, the model, the manufacturer, or a serial number of the handpiece, can be stored in the RFID transponder. This makes it possible to automate documentation.

For example, in a simple embodiment it is possible to use a read-only RFID transponder which can contain the above-mentioned data or also further data. In this situation the RFID transponder can, for example, be programmed by the manufacturer with the corresponding data. An autoclave or another device used for cleaning, sterilization or disinfection can, for example, read the data and store them in a database, in particular together with further potentially necessary data such as a date, a time, an operator or a substance used. Thus in case of doubt, for example, in the event of an inspection or in the case of an infection of a patient, the cleaning or sterilization process can be performed and demonstrated.

In an alternative embodiment it is also possible, for example, to use a RFID transponder which can also be written to. Thus not only fixed data, for example, the data mentioned above, can be stored for reading on the RFID transponder, but also dynamic data can be written to the transponder. This makes it possible for the further documentation already mentioned above to be performed on the transponder of the handpiece itself. This can take place, for example, in addition to documentation in a database or also as an alternative to documentation in a database.

The head may, for example, have receiving means for the tool which may also be designed for driving the tool. The drive motor is typically formed externally of the handpiece and can act on the handpiece via means such as, for example, a rotatable, typically flexible shaft or even by means of air pressure. In this case the drive motor typically imparts a rotary movement, which is then in turn transmitted to the tool. Within the handpiece this transmission typically takes place by means of the drivetrain. The transmission of rotational energy may be understood in particular as the transmission of a rotation.

The handpiece may be designed in particular to be autoclavable or designed in some other way in order to be mechanically cleaned, disinfected or sterilized. For example, a corresponding resistance can be provided against specific chemicals or against heat.

In an advantageous manner the plastic casing makes it possible that the RFID transponder can be accessed although it is arranged within the handpiece. Within the scope of the invention it has been recognized that known embodiments of handpieces which are encased with metallic material preclude the use of a RFID transponder in so far as this does not hinder the propagation of the radio waves necessary for reading the RFID transponder. Although this could be remedied by mounting the RFID transponder externally on the handpiece, it has been shown that in this case the substances used for cleaning and disinfecting typically damage RFID transponders and so prejudice the functional capability thereof. Thus the embodiment of a handpiece according to the invention offers a special advantage in so far as the RFID transponder can be read easily through the plastic casing, through which radio waves can propagate without problems, and is simultaneously protected against damage.

A preferred embodiment provides that between the RFID transponder and the hand rest area, in particular in axial direction of the longitudinal extension of the handpiece no element is arranged that further attenuates or reflects radio waves.

For this reason it is advantageously provided that no further casing made of metallic material is provided around the plastic casing or within the plastic casing, in particular not outside the RFID transponder. In other words the plastic casing is the only sheathing or delimitation of the handpiece. This applies in particular in the region of the RFID transponder. In particular a region in which there are no metallic parts should advantageously be provided from the outer face of the handpiece to the RFID transponder.

According to particular embodiments the RFID transponder is provided on the head, on the connecting piece or on the connector sleeve. Thus, advantageous access for reading can be ensured, taking into account the geometric, structural and material-related conditions.

According to a preferred embodiment, the connecting piece is of bent configuration like a knee, has a head piece facing the head and a connector piece facing the connector sleeve. In this case the head piece or the connector piece carries the RFID transponder. This corresponds to an embodiment which has proved ergonomic in practice.

The plastic casing is preferably shell-shaped. Thus, an advantageous enclosure of the interior of the handpiece can be achieved. In this case the plastic casing can be formed in particular in multiple parts, for example in two parts, so that two or more shells inter-engage and/or overlap one another. Thus, the manufacturing process can be simplified and the stability can be increased.

The plastic casing can preferably be designed as a sleeve or in a sleeve-like manner. This also enables an advantageous enclosure.

In a preferred embodiment the plastic casing also forms a handrest area on his surface. The plastic casing fulfils several tasks. It protects the RFID transponder and at the same time forms a hand rest area that may be designed and optimised, accordingly to impart to the user a pleasant grip sensation. Furthermore no additonal outer sleeve made of metal or the like is required, that would only increase manufacturing costs and weight.

According to one embodiment the plastic casing is used or formed on the handpiece as a supporting element. In this case it can serve in particular for support and/or mounting of the drivetrain.

According to one embodiment it is provided that the drivetrain is used or formed as a supporting element. In other words, this may mean that the drivetrain predetermines the shape and the plastic casing is substantially adapted to this shape.

The drivetrain can in particular have a particular number of rods, transmissions, bearings, gears and/or bevel gears. Such elements are typically provided with structures which fix the position of these elements relative to one another.

Rods which transmit a rotary movement can for example be supported and held in position by bearings. In this case, the bearings typically enable only a rotary movement, whereas they prevent translational movement. These bearings may for example be ball bearings which have a particularly low friction and correspondingly low wear.

A bevel joint can be used for example in order to transmit a rotary movement to a bent part of the handpiece. For this purpose it is typically necessary that usually two bevel gears of the bevel joint are held exactly in position relative to one another, so that respective teeth interengage.

Thus for the functioning of a drivetrain a specific arrangement of the elements thereof relative to one another is typically necessary. This arrangement should typically also be kept constant during the operation.

A skeleton which fixes the elements relative to one another can be provided, for example, for the arrangement of the elements of the drivetrain. This skeleton is typically made of rigid material, for example, metal. In particular the RFID transponder is advantageously arranged outside the skeleton in order to avoid impairment of the communication of the RFID transponder.

According to a further embodiment it is provided that a sleeve surrounding the drivetrain is used or formed as a supporting element. In other words, this may mean that the sleeve predetermines the shape and the plastic casing is substantially adapted to this shape.

The sleeve may in particular be a hollow sleeve. In particular it may be made of metal. Typically the RFID transponder is arranged outside the skeleton in order to avoid impairment of the communication of the RFID transponder.

The sleeve can enclose the drivetrain completely or partially, typically at least predominantly. This applies in particular with regard to the above-mentioned components of the drivetrain. Thus the drivetrain can be protected against mechanical or other damage.

By means of the sleeve it is also possible to use simple manufacturing processes for the application of the plastic casing. For example, the plastic casing can be sprayed on. Penetration of the material of the plastic casing into the drivetrain, which could lead to malfunction, is advantageously prevented by the sleeve.

In particular, bearings for the rotating elements of the drivetrain can be fastened to the sleeve. For example, a number of ball bearings, by which rods which transmit a rotary movement can be held so as to be rotatable, but fixed laterally, can be fastened in the sleeve.

In the cases just described, in which the drivetrain or a sleeve is used or formed as a supporting element, the plastic casing may be designed so that it is adapted to the predetermined shape. A particular strength of the plastic casing can be dispensed with. This for example enables the plastic casing to be formed completely or partially of a soft material which offers the user pleasant haptics.

According to one embodiment, the plastic casing is formed as a sprayed-on sleeve. According to a further embodiment the plastic casing is formed as a pulled-on sleeve. Such embodiments enable suitable manufacturing processes and advantageous characteristics of the handpiece.

According to particular embodiments, the plastic casing may be translucent, transparent or in color. This enables the recognition of the handpiece and the distinguishing of other handpieces, which avoids the use of an incorrect handpiece in critical situations such as, for example, the treatment of a tooth or an operation. Moreover, due to a transparent or translucent embodiment, checking units located in the handpiece can take place without having to take the handpiece apart.

The RFID transponder is preferably embedded in the plastic casing at least multilaterally, particularly preferably on all sides. This enables secure mounting of the RFID transponder and protection against damage or soiling.

According to a preferred embodiment, the RFID transponder is arranged on or at a metal surface of the handpiece which reflects the radio waves. Thus the reading can be simplified.

According to a preferred embodiment, the RFID transponder is arranged on the connecting piece, which is bent like a knee on the side which is at an obtuse angle. In this case, the side which is at an obtuse angle should be understood in particular to be the side or the part which is bent by comparison with the connector piece and/or subtends an obtuse angle with the connector piece. This enables reading at a short distance in typical cleaning equipment such as autoclaves when the head is arranged at the top and a reading means, for example in the form an antenna suitable for the purpose, is located at a level corresponding to the RFID transponder in the device.

The handpiece preferably has a media tube and/or an optical fiber. More preferably, the media tube and/or the optical fiber are arranged on the side of the knee-like bent connecting piece which forms a reflex angle. In this case, the side which forms a reflex angle should be understood in particular to be the side or the part which is bent by comparison with the head piece and/or subtends a reflex angle with the head piece. Substances such as water for rinsing or a disinfecting agent, for example, can be supplied to the tool by means of a media tube. By means of the optical fiber illumination is possible, which can, for example, illuminate the region of a human or animal body to be treated by means of the tool. For this purpose the optical fiber can be guided outwards at a suitable location so that the light can exit to the exterior.

The plastic casing is preferably formed of composite material of the PEEK (polyether ether ketone) type or silicone. These materials have proved advantageous in practice.

Polyether ether ketone (abbreviated to PEEK) is a thermoplastic plastic which is resistant to high temperatures and belongs to the polyaryl ether ketone group of substances. Its melting temperature is 335° C. Silicones are also temperature-resistant in temperature ranges above 200° C., and are likewise suitable for a thermal sterilization process. Moreover, the invention also covers all other thermoplastic plastics which have a melting temperature above 130° C., preferably above 150° C., particularly preferably above 180° C.

An external shape of the plastic casing is preferably provided in such a way that the handpiece is optimally comfortable to hold. For example, grooves can be formed for this purpose. A softer material can also be used externally. In particular, in the cases described above, in which the plastic casing has no significance in terms of support or shaping, the entire plastic casing can also be made of smooth material.

Furthermore the invention also covers the use of a RFID transponder for the purpose of automated recognition of a handpiece, in particular if this handpiece has a plastic casing.

### BRIEF DESCRIPTION OF THE DRAWINGS

In the drawings, the invention is illustrated schematically in particular in one exemplary embodiment. In the drawings:

FIG. 1a shows a plan view of a handpiece according to the invention in a first exemplary embodiment,

FIG. 1b shows a side view of the handpiece of FIG. 1a,

FIG. 2a shows a plan view of a handpiece according to the invention in a second exemplary embodiment, and

FIG. 2b shows a side view of the handpiece of FIG. 2a.

### DETAILED DESCRIPTION

In the drawings the same or corresponding elements are in each case designated by the same reference numerals and therefore are not described repeatedly unless this is expedient. The disclosures contained in the entire description can be transferred analogously to parts which are the same with the same reference numerals or the same component designations. Also, the positional details selected in the description, such as for example top, bottom, side, etc., relate to the drawing immediately described and illustrated and may be transferred to the new position in the event of a change in position. Furthermore, individual features or combinations of features of the different illustrated and described exemplary embodiments may also constitute separately independent or inventive solutions or solutions according to the invention.

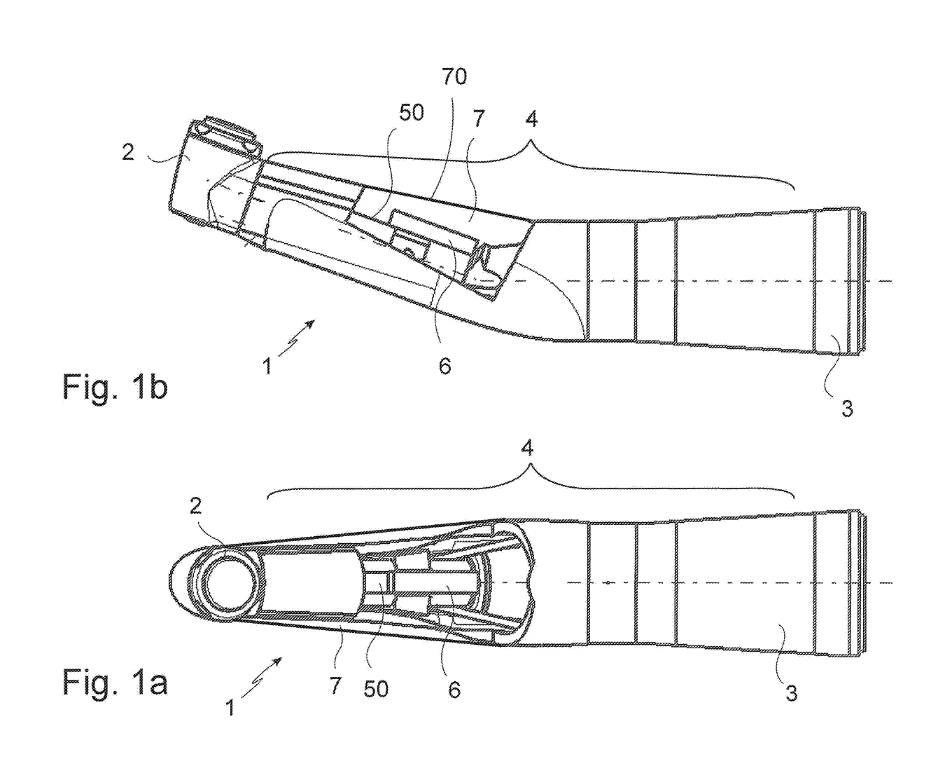

FIG. 1 shows a handpiece according to a first exemplary embodiment. In this case, FIG. 1a shows a plan view and FIG. 1b shows a side view.

The handpiece 1 has a head 2, a connector sleeve 3 as well as a connecting piece 4. The connecting piece 4 connects the head 2 to the connector sleeve 3. As shown, the connecting piece 4 is configured like a knee and slightly angled, wherein the part of the connecting piece 4 which is located nearer to the head 2 than to the connector sleeve 3 is at an obtuse angle to the part of the connecting piece 4 which is nearer to the connector sleeve 3.

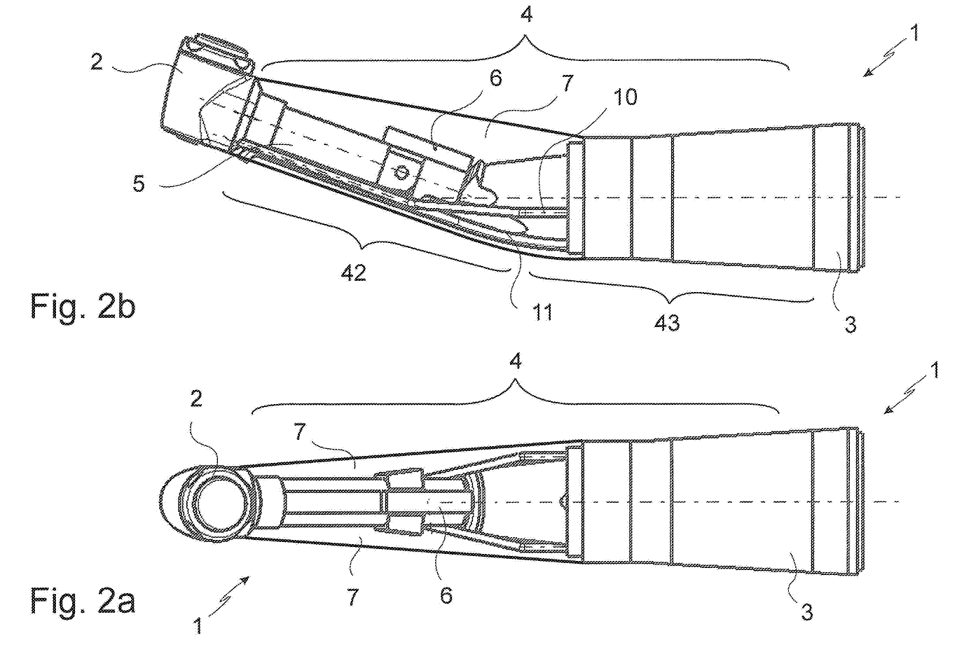

The head 2 is designed to receive and to hold a tool (not shown). Furthermore, it is designed to carry out a rotary movement onto the tool. Such a rotary movement is usually produced by means of an electric motor acting on the connector sleeve 3 and in the connecting piece 4 this movement is transmitted to the head 2. This is explained in greater detail below with reference to FIG. 2.

A metallic surface 50 on which an RFID transponder 6 is arranged is located inside the connecting piece 4. This region of the handpiece 1 is enclosed by a plastic casing 7. This casing is designed as a sleeve and is transparent. Thus the interior of the handpiece 1 is visible.

Data such as the manufacturer, the model, the date of manufacture and the serial number of the handpiece 1 are stored in the RFID transponder 6. The RFID transponder 6 can be simply read wirelessly by means of a corresponding reading device, wherein for this purpose radio waves are directed onto the RFID transponder 6 and are changed in a suitable manner by the RFID transponder. This is supported by the metal surface 50 which reflects the radio waves.

The RFID transponder 6 enables particularly simple automated sensing and unambiguous identification of the handpiece 1, which for example can significantly simplify the documentation of a cleaning process.

The plastic casing 7 at least partially forms the outer skin/hand rest area 70 of the handpiece 1 and determines the visual appearance and haptics thereof. Thus the handling and ease of operation can be optimized by suitable choice of the material of the plastic casing 7. Furthermore, the material is preferably selected so that it survives typical cleaning, disinfection and sterilizing operations undamaged. This generally involves resistance against the cleaning and disinfection fluids used as well as against increased temperature during sterilization. It may also be mentioned that the handpiece 1 and in particular the plastic casing 7 are designed to be autoclavable.

FIG. 2 shows a second exemplary embodiment of a handpiece 1. In this case FIG. 2a shows a plan view, whereas FIG. 2b shows a side view.

The handpiece 1 according to the second exemplary embodiment is of similar design to the handpiece 1 according to the first exemplary embodiment. In this case, FIG. 2b shows that the connecting piece 4 is subdivided into a head piece 42 and a connecting piece 43. In this case the head piece 42 is the section of the connecting piece 4 which is nearer to the head 2 than to the connector sleeve 3 and which is also connected to the head 2. On the other hand the connector piece 43 is the section of the connecting piece 4 which is nearer to the head 2 than to the connector sleeve 3 and which is also connected to the head 2.

As shown, the head piece 42 is angled by comparison with the connector piece 43. In this case the head piece 42 is at an obtuse angle to the connector piece 43. Thus the head piece 42 is the side of the connecting piece 4 which has an obtuse angle. Conversely the connector piece 43 forms a reflex angle.

Also the handpiece 1 according to the second exemplary embodiment has an RFID transponder 6 which is arranged at a similar location as that of the first exemplary embodiment. This also fulfils the same purpose as has already been described above with respect to the first exemplary embodiment.

In contrast to the first exemplary embodiment, however, the region of the connecting piece 4 which contains the RFID transponder 6 is not covered by a plastic casing made of transparent material and formed as a sleeve. On the contrary, this region is covered by a plastic casing 7 which is formed as a sprayed compound. In principle this is not transparent, but is shown as transparent in FIGS. 2a and 2b so that the components of the handpiece 1 are visible below the plastic casing 7.

Also, in the case of the first exemplary embodiment, the plastic casing 7 constitutes an outer skin of the handpiece 1 and defines the visual appearance and haptics thereof. It encloses the RFID transponder multilaterally and protects it against external influences.

Furthermore, a drivetrain 5 which is designed to transmit the rotation of the connector sleeve 3 to the head 2 is located below the plastic casing 7. A holder, which is not visible in the drawings, for a tool (not shown) is arranged in the head 2 and can then in turn transmit this rotation to the tool. This enables a tool inserted in the head 2 to be driven by means of a motor (not shown) which acts on the connector sleeve 3. In this case the drivetrain 5 has a skeleton which is not illustrated in greater detail and which holds individual parts of the drivetrain in a defined positional relationship relative to one another. Thus the shape of the handpiece 1, including the plastic casing 7, is predetermined. These statements apply correspondingly to the first exemplary embodiment.

Furthermore, it can be seen in FIG. 2b that a media tube 10 and an optical fiber 11 extend through the connecting piece 4. In this case the media tube 10 and the optical fiber 11 extend from the connector sleeve 3 to the head 2.

In the media tube 10 media such as, for example, water for rinsing or chemicals for disinfection or for influencing tissue, or also drugs, can be directed to the head 2. From there it can be passed on to the tool held in the head 2. Thus, for example, a rinsing function of the tool can be operated for dental purposes.

In the optical fiber 11 light can be directed from the connector sleeve 3 to the head 2, so that by means of a light (not shown) which is attached to the connector sleeve 3 or co-operates therewith it is possible to illuminate the head 2 or to light up the surroundings of the head 2. For example, suitable exit windows (not shown) for the optical fiber 11 can be provided, so that the light conveyed in the optical fiber 11 can exit at these exit windows. These exit windows can be arranged so that a region to be worked on using the tool held in the head 2 is illuminated. Thus, for example, the risk of faulty treatments can be reduced.

Both in the first and also the second exemplary embodiment no metallic component enclosing the RFID transponder is provided at least in the region of the connector piece 4 in which the RFID transponder is arranged. In particular there is no metal component present in the plastic casing 7. Thus the reading of the RFID transponder is considerably simplified, since shielding of reading radio waves by surrounding metal parts is avoided.

The claims filed now with the application and later are without prejudice for the achievement of more far-reaching protection.

If closer examination, in particular also of the relevant prior art, reveals that one or the other feature is favorable for the object of the invention, but is not crucially important, then of course a formulation will be sought which no longer includes such a feature, in particular in the main claim. Also such a sub-combination is covered by the disclosure of this application.

It should also be noted that the configurations and variants of the invention described in the various embodiments and illustrated in the drawings can be combined with one another in any way. In this case individual features or a plurality of features are interchangeable with one another in any way. These combinations of features are also disclosed.

The dependencies set out in the dependent claims refer to the further embodiment of the subject matter of the main claim by the features of the respective subordinate claim. However, these are not to be understood as a renunciation of the achievement of independent objective protection for the features of the dependent subordinate claims.

Features which have only been disclosed in the description or also individual features from claims which include a plurality of features can be incorporated into the independent claim/claims as features of essential significance to the invention in order to distinguish them over the prior art at any time, even when such features have been mentioned in connection with other features or also achieve particularly favorable results in connection with other features.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. Accordingly, this description is meant to be taken only by way of example, and not to otherwise limit the scope of this invention.

What is claimed is:

1. A dental or surgical handpiece comprising:
a head configured to receive a tool, and
a connector sleeve configured to couple the handpiece to a drive motor,
wherein the head and the connector sleeve are connected by a connecting piece and the connecting piece accommodates a drivetrain that transmits rotational energy from the connector sleeve into the head for driving the tool,
wherein the handpiece has an RFID transponder and a plastic casing delimiting the handpiece, and the RFID transponder is protected by the plastic casing.

2. The handpiece according to claim 1, wherein the RFID transponder is provided on the head, on the connecting piece, or on the connector sleeve.

3. The handpiece according to claim 1, wherein the connecting piece is bent an obtuse angle and has a head piece facing the head and a connector piece facing the connector sleeve, and the head piece or the connector piece supports the RFID transponder.

4. The handpiece according to claim 1, wherein the plastic casing is formed as a shell.

5. The handpiece according to claim 1, wherein the plastic casing is formed as a sleeve or sleeve shaped, or as a pulled-on tube.

6. The handpiece according to claim 1, wherein the plastic casing is used on the handpiece as a supporting element for supporting or bearing the drivetrain.

7. The handpiece according to claim 1, wherein the plastic casing is formed as a sprayed-on sheath.

8. The handpiece according to claim 1, wherein the plastic casing forms a handrest area on his surface.

9. The handpiece according to claim 1, wherein the plastic casing is translucent, transparent, or colored.

10. The handpiece according to claim 1, wherein the RFID transponder is embedded at least multilaterally in the plastic casing.

11. The handpiece according to claim 1, wherein the RFID transponder is arranged on or at a metal surface of the handpiece which reflects radio waves.

12. The handpiece according to claim 1, wherein the RFID transponder is arranged on the side of the connecting piece which forms an obtuse angle.

13. The handpiece according to claim 1, wherein the handpiece has a media tube and/or an optical fiber, and the media tube and/or the optical fiber is arranged on the side of the connecting piece which forms a reflex angle.

14. The handpiece according to claim 1, wherein the plastic casing is formed from composite material of the PEEK type or silicone.

15. The handpiece according to claim 1, wherein in an axial direction of the longitudinal extension between the RFID transponder and the handrest area, no element is provided that further attenuates or reflects radio waves.

16. The handpiece according to claim 10, wherein the RFID transponder is embedded on all sides in the plastic casing.
